(19) United States
(12) Patent Application Publication
(10) Pub. No.: US 2022/0380748 A1
(43) Pub. Date: Dec. 1, 2022

(54) PARTICLES FOR USE IN ACOUSTIC PROCESSES

(71) Applicant: FloDesign Sonics, Inc., West Springfield, MA (US)

(72) Inventors: Krishna N. Kumar, Wilbraham, MA (US); Bart Lipkens, Bloomfield, CT (US); Rui Tostoes, Granby, MA (US)

(21) Appl. No.: 17/753,750

(22) PCT Filed: Oct. 28, 2020
(86) PCT No.: PCT/US2020/057756

§ 371 Date: Mar. 14, 2022

Related U.S. Application Data
(60) Provisional application No. 62/927,070, filed on Oct. 28, 2019.

(51) Int. Cl. C12N 13/00; B01J 13/04; C12M 1/00

(52) U.S. Cl. CPC C12N 13/00; B01J 13/04; C12M 47/04

(57) ABSTRACT

Microparticles and nanoparticles made of various materials that are used in various configurations are disclosed. The particles may be perfluorocarbon droplets with a lipid coating. The particles may be used in an acoustic cell selection process. The droplets are highly acoustically responsive and can be retained against fluid flow by an acoustic field. Such particles can be used in the separation, segregation, differentiation, modification or filtration of a system.

### BACKGROUND

Acoustophoresis refers at least in part to the separation of materials using acoustics, such as acoustic standing waves or acoustic traveling waves. Acoustic waves, including standing or traveling waves, can exert forces on particles in a fluid when there is a differential in a parameter of the particles and the fluid that can be influenced by acoustics, including density and/or compressibility, otherwise known as the acoustic contrast factor. The pressure profile in a standing wave contains areas of locally reduced pressure amplitudes at standing wave nodes and locally increased pressure amplitudes at standing wave anti-nodes. Depending on, for example, their density and compressibility, the particles can be driven to the nodes or anti-nodes of the standing wave. Generally, the higher the frequency of the acoustic standing wave, the smaller the particles that can be manipulated.

At a micro scale, for example with structure dimensions on the order of micrometers, conventional acoustophoresis systems use acoustic chambers with a width dimension that is half or quarter wavelength, which at frequencies of a few megahertz are typically less than a millimeter in thickness, and operate at very low flow rates (e.g., μL/min). Such systems are not scalable since they benefit from extremely low Reynolds number, laminar flow operation, and minimal fluid dynamic optimization.

At the macro-scale, planar acoustic standing waves have been used in separation processes. However, a single planar wave tends to trap the particles or secondary fluid such that separation from the primary fluid is achieved by turning off or removing the planar standing wave. Planar waves also tend to heat the media where the waves are propagated due to the energy dissipation into the fluid that is involved with generating a planar wave and the planar wave energy itself. The removal of the planar standing wave may hinder continuous operation. Also, the amount of power that is used to generate the acoustic planar standing wave tends to heat the primary fluid through waste energy, which may be disadvantageous for the material being processed.

Cell selection/separation has been achieved by providing functionalized beads that have an affinity for a target cell or cellular material. The beads have a characteristic that permits their separation from a fluid typically containing other cells or cellular material. One approach uses beads with a ferro-magnetic characteristic, which allows their separation using magnetic fields.

In life science research and therapy, cells are sought to be manipulated for such purposes as separation or isolation. For example, chimeric antigen receptor (CAR) T-cells are developed as a therapy for certain types of cancers. CAR T-cell therapies have been developed where modified cells are isolated from a cell population using various techniques based on magnetic force, electrical force, gravitational force, microfluidics etc. In some applications, the cell of interest (in positive selection) is linked to a particle, such as a bead, that is functionalized with an affinity for the particular cell. The cell-bead complex is exposed to a force that can influence the bead. For example, a cell-magnetic particle complex may be exposed to a magnetic force that can influence the magnetic particle to permit the complex to be separated from other material with which the cell-magnetic particle complex is mixed. In the case of positive selection of cells, the cell-magnetic particle complex, or target material, may be retained by the magnetic force, while other, non-target material is not retained. In the case of negative selection of cells, other material than the target cells are bound to a bead so that the target cells are not retained by the magnetic force and can thus be separated from the other material.

The aim of using a force modality to separate cellular material is to obtain high purity and increase the recovery of the desired cells. Available techniques have challenges in that it is difficult or impractical for these processes to be scaled up. Moreover, some techniques have known detrimental effects on the health of cells. For example, one technique uses of magnetic beads to isolate desired cells from other material, which is often other types of cells. In this method, a mixture of cells and cell-magnetic bead complex is passed thru very narrow column/channels of diameter less than 1 mm and the beads in the column are exposed to a strong magnetic field. Because the size of the channels is relatively small, freely flowing cells are exposed to very high shear fluidic forces that can be damaging and detrimental to the health of cells. The cell-bead complex which is held at the wall of the column due to exposure to the strong magnetic field experiences even higher shear stress due to a high magnetic force that is typically normal to the direction of flow in the column. Another drawback of this approach is that flow rate is severely limited, which increases processing time and limits the ability of the technique to be scaled up. In addition, one such technique uses nanometer sized magnetic beads, which can possibly be internalized with the cells, which can be problematic for cell therapy treatments.

### SUMMARY

In various examples herein, materials and methods are disclosed for acoustically responsive particles that can be linked to cellular material and influenced by an acoustic field. Materials and methods are disclosed for manufacture of the particles, including functionalizing the particles to link to particular cell types or cellular material. As used herein, the term “particles” may be used generally interchangeably with the terms “beads” and/or “droplets.” The particles are placed within an acoustophoretic device, and an ultrasonic acoustic transducer is used to generate an acoustic field that can block, concentrate, trap, move and/or generally manipulate the particles as desired.

As discussed herein, particles in the micrometer or nanometer range are manipulated with acoustic fields, which may be generated via ultrasonic acoustic waves, including traveling and/or standing waves. The acoustic fields influence the particles to achieve blocking, trapping, concentration, transport and/or any other type of manipulation that the acoustic fields can impose on the particles. The influence of the acoustic fields on the particles may be enhanced by fluid dynamics and particle physics. For example, concentrating particles in a certain area using acoustic fields may create a boundary condition at which a pressure differential is formed. Such a pressure differential may enhance a concentration or separation effect generated by the acoustic field.

The particles discussed herein may be used for cell isolation. For example, the particles may be used for isolation of T-cells or chimeric antigen receptor (CAR) T-cells for CAR T cell therapy applications. The particles may also be used for other types of cell and gene therapy applications, such as, for example, genetically modified CD34+ cell therapies.

In some example implementations, a bead is composed of a perfluorocarbon droplet with a lipid coating. The bead is manufactured by preparing a lipid compound, combining a perfluorocarbon with the lipid compound and agitating the combination. In some examples, agitation is performed using centrifugation of the combination to obtain the beads. The beads may be used in a cell selection process by functionalizing the beads to have an affinity or linkage that can bind the beads to desired cells in a fluid that also entrains other cells or cellular material. The bead-cell complex is exposed to an acoustic field to manipulate the complex, such as by retaining the complex in a certain region.

The particles may be constructed to include a liquid core; and a lipid shell encapsulating the liquid core. The liquid in the liquid core may be composed of a perfluorocarbon. The perfluorocarbon may be perfluoropentane, perfluorohexane, perfluorooctane, perfluorooctyl bromide, perfluorodichlorooctane, or perfluorodecalin.

The lipid shell can be formed from dipalmitoylphosphatidylcholine (DPPC), 1,2-palmitoyl-phosphatidic acid (DPPA), a lipid-polyethylene glycol conjugate, or a complex of a lipid with albumin. The lipid shell can be functionalized with, for example, streptavidin, biotin, avidin, desthiobotin, an aptamer, an oligonucleotide and/or an antibody, collectively referred to herein as a linker, either in part or in whole. The lipid shell may completely or partially encapsulate the liquid core.

A process known as acoustic droplet vaporization (ADV) can be used to generate a phase shift of the liquid core of such particles from liquid to gas using an acoustic wave. The vapor pressure of the liquid is a function of temperature, and is not necessarily based upon the liquid chemistry. Any liquid that has a normal boiling point near or below the body temperature can be used for these processes. Perfluorocarbons may be utilized in these processes because of their low toxicity and high contrast factor.

A spacer may be placed in between the particle and the linker. The spacer can be implemented as a polyethylene glycol (PEG) molecule. The PEG molecule may permit less charge interference from the particle when materials are binding to the functionalized molecule on the surface of the particle.

In some example implementations, an acoustically responsive bead/particle/droplet for cell isolation using acoustic waves is provided. The particle may have a liquid core that is composed of a perfluorocarbon such as n-perfluorohexane/n-perfluoropentane/n-perfluoroheptane/perfluoro-octyl bromide or combinations of these perfluorocarbons. The liquid core is encapsulated, in whole or in part, with a lipid compound. The lipid compound may be provided with a linker or a ligand that can target cells, antibodies, viruses, or aptamers. These lipid compound may be composed of one or more of PEG 40 Stearate, dipalmitoylphosphatidylcholine (DPPC), 1,2-palmitoyl-phosphatidic acid (DPPA), 1,2-dipalmitoyl-sn-glycero-3-phosphoethanolamine (DPPE), 1,2-distearoyl-sn-glycero-3-phosphoethanolamine (DSPE), DSPC 1,2-distearoyl-sn-glycero-3-phosphocholine, DSPC, PBS buffer, propyleneglycol, glycerol, DSPE-mPEG(2000) 1,2-distearoyl-sn-glycero-3-phosphoethanolamine-N-[methoxy(polyethylene glycol)-2000] (ammonium salt), DSPE-PEG(2000) Biotin 1,2-distearoyl-sn-glycero-3-phosphoethanolamine-N-[biotinyl(polyethylene glycol)-2000] (ammonium salt), DSPE-PEG(2000) Desthiobiotin 1,2-distearoyl-sn-glycero-3-phosphoethanolamine-N-[desthio-biotinyl(polyethylene glycol)-2000] (ammonium salt), Polyoxyethylene (40) stearate, DSPE-PEG(2000) Maleimide 1,2-distearoyl-sn-glycero-3-phosphoethanolamine-N-[maleimide(polyethylene glycol)-2000] (ammonium salt), or a functionalized lipid-glycol conjugate, here labeled as DSPE-PEG5000-BIOTIN, as examples. The ligand or linker may be composed of NeutrAvidin, Avidin, StreptAvidin, CaptAvidin, biotin, desthiobotin, an aptamer, an oligomer, such as an oligonucleotide and/or an antibody.

The particle may be manufactured by combining an aqueous lipid solution and perfluorocarbons which may be homogenized/sonicated/membrane emulsified/mechanical agitation (vial mixing) to produce the desired size distribution (based on application). A downstream centrifugation is may be used for narrowing the size of the particle distribution or for washing purposes. The particle size distribution depends on the method incorporated to manufacture the droplet/bead/particle. The manufactured particle size may be in the range of from about 400 nm to about 300 microns.

The droplet/particle/bead may be incubated with one or more different types of ligands or linkers, such as NeutrAvidin/StreptAvidin/CaptAvidin depending on the application. The final droplet/particle/bead is used for further applications, such as cell selection or sorting in an acoustic device. The final droplet/bead/particle solution may have BSA/HSA or some stabilizer/surfactant in the aqueous part of the solution.

The droplet/particle/bead may be used for positive or negative selection of cells. In some examples, The droplet/particle/bead functionalized with desthiobiotin in the encapsulation is used for positive selection of cells. The droplet/particle/bead could be eluted from a complex by the addition of biotin buffer. The functionalization of the droplet/particle/bead can be formed as a reversible link for binding with a cell. For example, a biotin-Neutravidin bond can be separated to detach the droplet/particle/bead from the cell.

According to an example implementation, a method for manufacturing particles is provided that includes preparing a lipid compound, combining a perfluorocarbon with the liquid compound, and agitating the combination. The agitation may be achieved by a combination of one or more of centrifugation, sonication, homogenization or mechanical agitation. The agitation may be implemented to achieve a predetermined particle size distribution. The particle size distribution may be in a range of from about 400 nm to about 300 microns. The particle may be manufactured by combining different lipids in a sequence based on a characteristic of each lipid, such as by preparing a solution with a lipid solvent, heating the solution and adding the different lipids to the solution in order of solubility.

The lipid compound may include one or more of DPPA, DPPC, DSPC, PEG40 Stearate, DSPE-mPEG(2000), DSPE-PEG(2000)-Biotin, DSPE-PEG-5000-Biotin, DSPE-PEG(2000)-Desthiobiotin, PBS buffer, glycerol, propyleneglycol, or DSPE-PEG(2000)-Maleimide. The perfluorocarbon may be one or more of perfluoropentane, perfluorohexane, perfluorooctane, perfluorooctyl bromide, perfluorodichlorooctane, or perfluorodecalin. The particle may be functionalized with a linker, such as a reversible linker, including one or more of Avidin, Neutravidin, Streptavidin, Captavidin, biotin, desthiobiotin, an antibody, an aptamer or an oligomer. The particle may include a stabilizer or surfactant, which may be in the liquid core portion.

The particle may be used in a method for separating target particles from a fluid, where the method includes receiving functionalized particles in the fluid in a chamber, receiving target particles in the chamber, permitting the target particles to bind with the functionalized particles, and applying an acoustic wave to the chamber to influence the functionalized particles to be collected or blocked by the acoustic wave.

The developed particles yield very high purity and recovery of cells. The acoustic affinity particle in the presence of acoustic field performed well at all the scales in a reasonable amount of time, without compromising the output and health of cell.

In this work an acoustic affinity particle was developed for the purpose of isolating cells. As the cell separation is based on acoustic force, so a biocompatible liquid with high compressibility such as perfluorohexane (PFH) was selected as core of the particle/droplet. Phospholipids were used as an emulsifier. For cell targeting, one of the phospholipids is biotinylated and biotin-neutravidin interaction is used for targeting. In applications where elution of cell is desired from the perfluorohexane droplet, the regular biotin molecule in the encapsulation is replaced with desthiobotin. The droplet manufacturing process was designed to achieve a size distribution which was responsive to the acoustic wave and at the same time should have sufficient surface area for binding with the cells. The binding and separation of cells using PFH droplets were investigated for both negative and positive selection applications. In the test cases the PFH droplets in the presence of acoustic wave yielded high purity and recovery of the target cells. Desthiobiotin may be conjugated to the droplets. In positive selection of cells, the droplets are modified to elute from the cell-antibody-droplet complex and the elution technique resulted in high elution efficiency without any detrimental effect on the cells.

An acoustic affinity particle is developed for the purpose of isolating cells. An acoustically responsive liquid such as perfluorohexane (PFH) is used as core and Phospholipids as an emulsifier. The droplet manufacturing process was designed to achieve a size distribution suitable for binding and good acoustic response. Biotin-neutravidin interaction was used for targeting. For positive selection of cells where elution is desired, the regular biotin molecule in the encapsulation is replaced with desthiobotin. Both negative and positive selection cell isolation were performed in the presence of acoustic wave and it yielded high purity and recovery of the target cells.

The developed particles are intended to be used for cell isolation using acoustic wave in various chimeric antigen receptor (CAR) T cell therapy applications and cell and gene therapy applications such as genetically modified CD34+ cell therapies.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a graph showing force imposed on a particle in a standing wave field.

FIG. 2 is a graph showing force imposed on a particle in a traveling wave field.

FIG. 3 is a schematic illustration of a particle comprising a liquid core and a lipid shell.

FIGS. 4 and 5 are graphs showing size distribution of particles.

FIGS. 6 and 7 are graphs showing fluorescence intensity versus particle count.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that permit the presence of other ingredients/components/steps than those specifically named. However, such description should be construed as also describing compositions, articles, or processes as “consisting of” and “consisting essentially of” the enumerated ingredients/components/steps, which allows the presence of only the named ingredients/components/steps, along with any impurities that might result therefrom, and excludes other ingredients/components/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

It should be noted that many of the terms used herein are relative terms. For example, the terms “upper” and “lower” are relative to each other in location, e.g. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, e.g. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, e.g. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, e.g. the surface of the earth. The terms “upwards” and “downwards” are also relative to an absolute reference; upwards is always against the gravity of the earth.

The present application refers to “the same order of magnitude.” Two numbers are of the same order of magnitude if the quotient of the larger number divided by the smaller number is a value of at least 1 and less than 10.

The term “virus” refers to an infectious agent that can only replicate using a living cell, and otherwise exists in the form of a virion formed from a capsid that surrounds and contains DNA or RNA, and in some cases a lipid envelope surrounding the capsid.

The term “crystal” refers to a single crystal or polycrystalline material that is used as a piezoelectric material.

The present disclosure refers to “microparticles.” This term refers to particles having an average particle diameter of 1 micrometer (μm) to 1000 μm.

The present disclosure refers to “nanoparticles.” This term refers to particles having an average particle diameter of 1 nanometer (nm) to less than 1000 nm.

Some of the materials discussed herein are described as having an average particle diameter. The average particle diameter is defined as the particle diameter at which a cumulative percentage of 50% (by volume) of the total number of particles are attained. In other words, 50% of the particles have a diameter above the average particle size, and 50% of the particles have a diameter below the average particle size. The size distribution of the particles may include a Gaussian distribution, with upper and lower quartiles at 25% and 75% of the stated average particle size, and all particles being less than 150% of the stated average particle size. Any other type of distribution may be provided or used. It is noted that the particles do not have to be spherical. For non-spherical particles, the particle diameter is the diameter of a spherical particle having the same volume as the non-spherical particle.

Particles may be described herein as having a “core” and “shell” structure. The term “particle” is meant to refer to any type of individual structure that may be suspended in a fluid such as a liquid or gas and may be in any phase, e.g., solid, liquid or gas and combinations thereof.

“Organic” and “inorganic” materials are referred to herein. For purposes of the present disclosure, an “organic” material is made up of carbon atoms (often with other atoms), whereas an “inorganic” material does not contain carbon atoms.

The present disclosure may refer to temperatures for certain process steps. In the present disclosure, the temperature usually refers to the temperature attained by the material that is referenced, rather than the temperature at which the heat source (e.g. furnace, oven) is set. The term “room temperature” refers to a range of from 68° F. (20° C.) to 77° F. (25° C.).

In cell processing, such as might be used in developing a CAR T cell therapy, the desired cells are isolated from the main population using various techniques based on magnetic force, electrical force, gravitational force or microfluidics, to name a few. In some applications, the cell of interest (in positive selection) is attached to a particle/bead using an antibody or aptamer or oligomer and the cell-bead complex is flowed thru a region/chamber exposed to a force which is based on the nature of the particle. For example, a cell-magnetic particle complex may be passed through a chamber/column that is exposed to a magnetic force. In case of positive selection of cells, the retained cells in the chamber are the desired cells whereas in negative selection, the cells which are not retained in the chamber are the desired cells.

In acoustic cell processing, the desired cells are entrained with other cellular material or cells in a fluid from which the desired cells are sought to be isolated or separated. The cells of interest (in positive selection) are attached or bound to an acoustically responsive bead using a linking mechanism that may include an antibody, aptamer, oligomer, or any other suitable cell-bead linking mechanism. The cell-bead complexes are flowed with the material with which they are entrained through a region/chamber where they are exposed to an acoustic field that influences the beads. In the case of positive selection of cells, the desired cells are retained (via the beads) by the acoustic field, whereas in the case of negative selection, the desired cells are not retained by the acoustic field.

The acoustic separation/isolation of cells using the beads discussed herein is advantageous over other techniques since high purity results as well as a high percentage recovery of the desired cells can be obtained while maintaining cell health and integrity. In addition, acoustic cell processing can be scaled up, while having little or no detrimental effect on the health of the cells. For example, the cells and cell-bead complexes experience little or no additional shear stress due to the manipulation by the acoustic field. Moreover, because the acoustic cell processing discussed herein is a macro process, it is not severely limited in flow rate, and can have higher throughput and shorter processing times than conventional techniques. Furthermore, the beads discussed herein are non-toxic to humans, and are therefore much more appealing for use in therapeutic manufacturing processes than conventional magnetic beads.

The present disclosure relates to particles that are used in conjunction with acoustophoretic devices that include an ultrasonic transducer. The ultrasonic transducer generates acoustic waves that can be used to manipulate particles in various ways. For example, the acoustic waves can be used to block particles from movement into a certain region, to move particles to and/or retain particles at a desired location or trajectory. The particles can be microparticles or nanoparticles, as desired. The particles are acoustically responsive. The particles may be referred to as beads or droplets, each of which terms may be used interchangeably herein.

As discussed above, the particles are generally microparticles or nanoparticles. The particles may be spherical in shape or may vary, such as, for example, the particles could be ellipsoidal or elongated along a longitudinal axis. For example, making particles out of multiple different layers can be used to obtain both a desired density and a desired acoustic contrast factor, or to obtain a desired behavior or interaction for the particle. The particles may be composed of perfluorocarbons (PFCs), which are highly acoustically responsive.

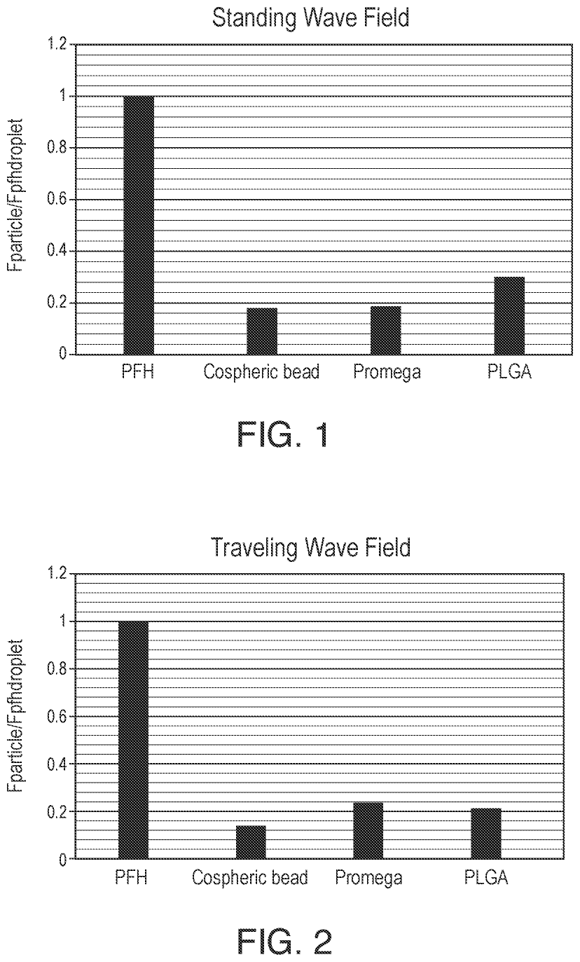

Equation 1 presents an analytical expression for the acoustic radiation force F_{R} on a particle in a fluid suspension in a planar standing wave. The acoustic contrast factor, X (equation 2), for a PFC droplet is negative, which means it will go to pressure antinodes unlike most of the commercially available beads which go to pressure nodes in an acoustic standing wave field. The acoustic contrast factors of perfluorohexane (PFH), Cospheric beads, Promega beads, and PLGA are −0.97, 0.18, 0.18, and 0.3 respectively. FIG. 1 shows the comparison of magnitude of force on beads of different materials with respect to PFH droplets. FIG. 1 shows that for the same size and excitation parameters, the PFH droplets are acoustically more responsive than other, commercially available beads. The high acoustic response can be attributed to the low speed of sound in PFH.

$$F \text{?} = \frac{3 π {P}^{2} \text{?} V \text{?} B \text{?}}{2 λ} X sin ( 2 kx ) \qquad ( 1 )$$

$$X = \frac{1}{3} [ \frac{5 ρ \text{?} - 2 ρ \text{?}}{2 ρ \text{?} + ρ \text{?}} - \frac{β \text{?}}{β \text{?}} ] \qquad ( 2 )$$

$$\text{?} \text{indicates text missing or illegible when filed}$$

Where: P₀ is Pressure amplitude, Vₚ is Volume of the particle, β_{f} is Compressibility of fluid, λ is Wavelength, k is Wavenumber, ρₚ is Density of particle, ρ_{f} is Density of fluid and X is acoustic contrast factor.

Equation 3 presents an analytical expression for the acoustic F_{R} radiation force on a particle in a fluid suspension in a planar travelling wave. In a travelling wave the force acts along the direction of the wave propagation. The expression shows that the force primarily depends on the density of the particle.

$$F \text{?} = \frac{6 \sqrt{2 π} {P}^{2} \text{?} {( 1 - \frac{ρ \text{?}}{ρ \text{?}} )}^{2} {R}^{2} \sqrt{\text{?}}}{ρ \text{?} {( 2 + \frac{ρ \text{?}}{ρ \text{?}} )}^{2} \text{?}} \qquad ( 3 )$$

$$\text{?} \text{indicates text missing or illegible when filed}$$

FIG. 2 shows a comparison of the magnitudes of acoustic forces on beads made of different materials with normalized to PFH droplets in an acoustic travelling wave field. FIG. 2 shows that for the same size and excitation parameters, the PFH droplets are acoustically more responsive than commercially available beads. The high acoustic response here can be attributed to the high density of liquid perfluorohexane.

In general, the particles of the present disclosure may be manipulated with acoustic fields that can be generated with acoustic waves, which can be standing waves or traveling waves. The acoustic fields can be generated to form a pressure rise near an interface region that creates a barrier to the particles.

The acoustic devices discussed herein may operate in a multimode or planar mode. Multimode refers to generation of acoustic waves by an acoustic transducer that create acoustic forces in three dimensions. The multimode acoustic waves, which may be ultrasonic, are generated by one or more acoustic transducers, and are sometimes referred to herein as multi-dimensional or three-dimensional acoustic standing waves. Planar mode refers to generation of acoustic waves by an acoustic transducer that create acoustic forces substantially in one dimension, e.g. along the direction of propagation. Such acoustic waves, which may be ultrasonic, that are generated in planar mode are sometimes referred to herein as one-dimensional acoustic standing waves.

The acoustic devices may be used to generate bulk acoustic waves in a fluid/particle mixture. Bulk acoustic waves propagate through a volume of the fluid, and are different from surface acoustic waves which tend to operate at a surface of a transducer and do not propagate through a volume of a fluid.

The acoustic transducers may be composed of a piezoelectric material. Such acoustic transducers can be electrically excited to generate planar or multimode acoustic waves. The three-dimensional acoustic forces generated by multimode acoustic waves include radial or lateral forces that are unaligned with a direction of acoustic wave propagation. The lateral forces may act in two dimensions. The lateral forces are in addition to the axial forces in multimode acoustic waves, which are substantially aligned with the direction of acoustic wave propagation. The lateral forces can be of the same order of magnitude as the axial forces for such multimode acoustic waves. The acoustic transducer excited in multimode operation may exhibit a standing wave on its surface, thereby generating a multimode acoustic wave. The standing wave on the surface of the transducer may be related to the mode of operation of the multimode acoustic wave. When an acoustic transducer is electrically excited to generate planar acoustic waves, the surface of the transducer may exhibit a piston-like action, thereby generating a one-dimensional acoustic standing wave. Compared to planar acoustic waves, multimode acoustic waves exhibit significantly greater particle trapping activity on a continuous basis with the same input power. One or more acoustic transducers may be used to generate planar and/or multi-dimensional acoustic standing waves. In some modes of operations, multimode acoustic waves generate an interface effect that can hold back or retain particles of a certain size, while smaller particles can flow through the multimode acoustic waves. In some modes of operation, planar waves can be used to deflect particles at certain angles that are characteristic of the particle size.

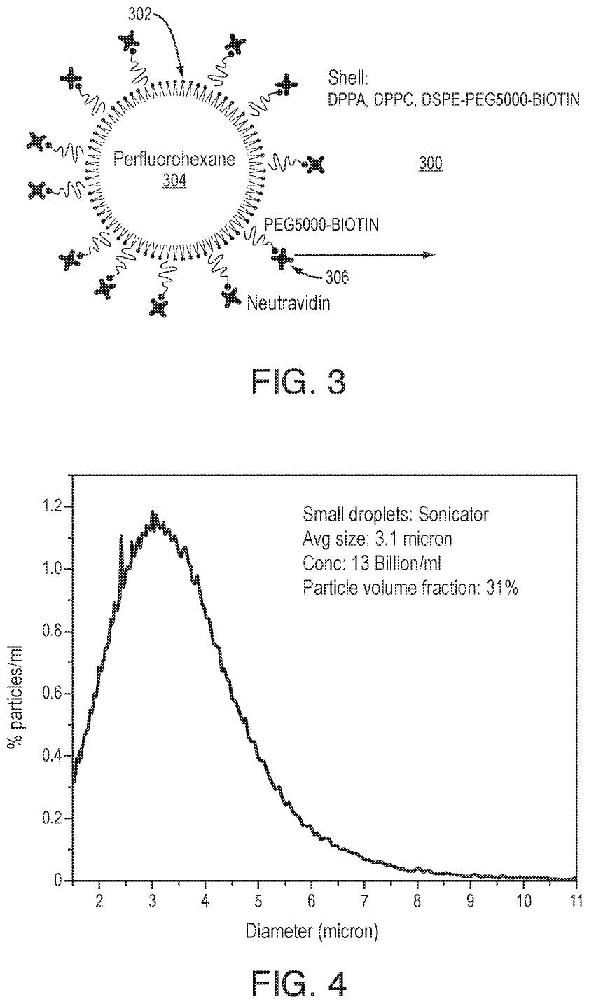

Discussed herein are PFC beads, processes for their manufacture, and methods for cell selection using the beads. Examples using perfluorohexane (PFH) beads are presented with techniques for cell targeting that is achieved using a biotin-neutravidin non-covalent interaction. The liquid perfluorohexane (PFH) core droplets are encapsulated with biotinylated-lipids with bound Neutravidin. FIG. 3 shows a schematic of the perfluorohexane core droplets.

The acoustic-based cell sorting is performed in an acoustic standing wave field, such as the multidimensional acoustic standing wave technology developed by FloDesign Sonics, Inc. in U.S. Pat. No. 9,822,333 to Lipkens, et al. The PFC liquids which were used to synthesize the core in the droplets have unique physical properties. The salient properties of PFC liquids are listed as: denser than water, low viscosity, low surface tension, high capacity to absorb oxygen, low speed of sound with respect to water, high chemical inertness, and biocompatibility. Table 1 shows the physical and acoustic property of the PFC liquids which were explored for droplet manufacturing.

TABLE 1
Physical and acoustic properties of PFCs at room temperature.

| | | | | B.P | |
|---|---|---|---|---|---|
| Compound | Formula | (kg/m3) | (m/s) | (° C.) | Compressibility |
| PFOB (Perfluorooctyl | C₈F₁₇Br | 1920 | 630 | 141 | 13.12 × 1010 |
| bromide) | | | | | |
| PFH | C₆F₁₄ | 1670 | 548 | 57 | 19.93 × 1010 |
| (Perfluorohexane) | | | | | |
| PFP | C₅F₁₂ | 1600 | 477 | 29 | 27.46 × 10¹⁰ |
| (Perfluoropentane) | | | | | |
| Cell | | 1060 | 1600 | | 3.68 10¹⁰ |

Perfluorocarbon (PFC) liquids have high compressibility, therefore they are suitable candidate for design of acoustically responsive particles. Perfluorocarbons (PFCs) are chemically inert compounds and have multiple biomedical applications. Their emulsion is used as an artificial blood substitute (Biro, Blais, & Rosen, 1987; Moore & Clark Jr, 1978; Yokoyama, Yamanouchi, Murashima, & Tsuda, 1981), acoustic contrast agents in molecular imaging (Lambert & Jablonski, 1997), and MRI contrast agents (Diaz-López, Tsapis, & Fattal, 2010) etc. Other biomedical applications of fluorocarbons include lung surfactant replacement (Clark Jr, 1998; Sekins, Shaffer, & Wolfson, 1996) and ophthalmologic aids (Vidne et al., 2018). Perfluorocarbons are also being employed to facilitate respiratory gas supply to cells (Goh, Gross, Simpson, & Sambanis, 2010; Ju & Armiger, 1992) and, in some systems, to improve biomass production and yields of commercially-important cellular products. Animal (including human) and plant cells have also been cultured at the interface between PFC liquids and aqueous culture medium. The ability of PFC liquids to dissolve respiratory gases has attracted much interest from clinicians and biotechnologists.

Some of the commercial products based on these characteristics of PFC are Fluosol (Fluosol-DA, Green Cross Corp., Osaka, Japan, and Alpha Therapeutic, Los Angeles, Calif., USA), Oxypherol (Fluosol-43, Green Cross Corp. and Alpha Therapeutic), Perftoran (Ftorosan, OJCS SPF Perftoran Russian, Moscow, Russia)), Oxygent (AF0144, Alliance Pharmaceutical Corporation, San Diego, Calif., USA), Oxyfluor (HemaGen/PFC, St. Louis, Mo., USA) and Oxycyte (Oxygen Biotherapeutics, Inc., formerly Synthetic Blood Int., Costa Mesa, Calif., USA). Definity (Lantheus Medical Imaging, N. Billerica, Mass.) is a US Food and Drug Administration (FDA) approved ultrasound contrast agent with lipid encapsulation and perfluorobutane gas core. It is used for cardiovascular imaging in the USA.

In some example implementations, the particles are of a core-shell structure, with a liquid core encapsulated by a lipid shell. For example, the liquid in the liquid core is a perfluorocarbon (PFC). The term “perfluorocarbon”, as used in the present disclosure, refers to molecules in which all of the hydrogen atoms have been replaced with a halogen, and a majority of the halogen atoms are fluorine atoms. For purposes of the present disclosure, “halogen” refers to fluorine, chlorine, and bromine. Specific examples of PFCs include perfluoropentane (PFP), perfluorohexane (PFH), perfluorodichlorooctane (PFDCO, C8F16Cl2), perfluorooctane (PFO), perfluorooctyl bromide (PFOB, C8F17Br), or perfluorodecalin (PFD, C10F18).

These PFC liquids have unique properties. The PFC liquids are denser than water, have low surface tension and have low viscosity. The PFC liquids also have a high capacity to absorb oxygen and nitrogen. Perfluorocarbon liquids have a low speed of sound, are highly chemically inert, and are biocompatible. Table 2, below, shows various physical and acoustic properties of various PFC liquids which may be used in particles, along with other polymers for comparison. It is noted that the compressibility of the PFC liquids is very high compared to biological cells.

TABLE 2

| | | Speed | | | | | |
|---|---|---|---|---|---|---|---|
| | | of | Boiling | | Specific | Surface | |
| | Density | Sound | Point | Contrast | Gravity | Tension | |
| Compound | (kg/m³) | (m/s) | (° C.) | Factor | (g/mL) | (mN/m) | Compressibility |
| PFP | 1600 | 477 | 29 | −1.59 | 1.6 | 9 | 27.46 × 10¹⁰ |
| (Perfluoro pentane) | | | | | | | |
| PFH | 1670 | 548 | 57 | −1.44 | 1.63 | 12 | 19.93 × 10¹⁰ |
| (Perfluoro hexane) | | | | | | | |
| PFOB | 1920 | 630 | 141 | −0.55 | 1.9 | 16 | 13.12 × 10¹⁰ |
| (Perfluoro octyl | | | | | | | |
| bromide) | | | | | | | |
| PMMA | | 2700 | | 0.299 | 1.18 | | |
| Polystyrene | | 2350 | | 0.22 | 1.06 | | |
| Cell | 1060 | 1600 | | | | | 3.68 × 10¹⁰ |

Specific examples of lipids that can be used to form the lipid shell include dipalmitoylphosphatidylcholine (DPPC), 1,2-palmitoyl-phosphatidic acid (DPPA), 1,2-dipalmitoyl-sn-glycero-3-phosphoethanolamine (DPPE), and 1,2-distearoyl-sn-glycero-3-phosphoethanolamine (DSPE). These lipids can also be used in a lipid-polyethylene glycol conjugate, or a complex of a lipid with albumin (such as bovine serum albumin or human serum albumin). The lipid shell can be functionalized with streptavidin, biotin, desthiobotin, avidin, an antibody, an aptamer, an oligonucleotide and/or other functionalized moieties. The lipid shell is used to attach the particle to another molecule, and for protection of the liquid core.

This structure is illustrated in FIG. 3. The particle 300 is made of a lipid shell 302 that surrounds a liquid core 304, in this example perfluorohexane. The shell can be made of DPPA, DPPC, DSPC, DSPE-PEG2000 or a functionalized lipid-glycol conjugate, here labeled as DSPE-PEG5000-BIOTIN. Also illustrated is Neutravidin, an avidin derivative 306 that binds to the biotin of the lipid shell.

A number of manufacturing or synthesis techniques are presented herein. According to one example implementation, a lipid blend is created, and the Perfluorohexane (PFH) liquid is dispersed therein by different methods depending upon the size of droplets/beads desired for the application. The lipid blend may include DSPC, PEG40 Stearate, DSPE-mPEG-2000, DSPE-PEG-2000-Biotin, DSPE-PEG-5000-Biotin, PBS buffer, propyleneglycol and glycerol. PFH and a lipid solution are mixed using a homogenizer to generate the droplets. The droplets obtained after homogenization is highly polydisperse. A downstream centrifugation protocol is performed to obtain a desired droplet size. After the droplets are manufactured, they are incubated with a desired quantity of Neutravidin and two steps of wash are performed to remove free Neutravidin.

In another example implementation, phospholipids are used as an emulsifier/surfactant. The hydrophile-lipophile balance (HLB) number for the lipid formulation used here is 13.53. The HLB number gives an indication that the emulsion formed here should be oil in water emulsion. Since the droplets will be attached to cells, a biotinylated lipid is included in the lipid formulation. In this example, the attachment of the droplets to cells is done with non-covalently linked Neutravidin. Other methods and techniques are described herein to make different size ranges of droplets. The exact composition and details of the lipids are provided below in Tables 3 and 4. The desired lipid blend is created, and the PFC liquid is dispersed by different methods depending upon the size of droplets/beads desired for the application. To create small size droplets ultrasonic agitation was used. For making larger droplets, a homogenizer is used to agitate the liquid mixture. The lipid solution preparation is an important part of the synthesis process.

In some examples, the procured lipids are stored in the freezer at −20 deg C. For synthesis of droplets, lipids are taken out of the freezer and left at room temperature for 20 minutes. The thawing at room temperature for 20 minutes is done to bring the lipids from the solid frozen state, to gel state. It is recommended to bring the lipids to liquid state during the emulsification process. Lipids do not dissolve in water, so propylene glycol is used to dissolve the lipids. It is recommended to not dissolve all the lipids at once in the propylene glycol. Combining all the lipids at one time may result in formation of white clumps in the solution, which may be difficult to dissolve. The order of solubility of the lipids is used, for example, as the least soluble lipid is dissolved first in the propylene glycol and so on. It is to be noted that solubility is a function of temperature of the solution. The solution is desirably maintained at a temperature which is above the transition temperature of the lipids. At the transition temperature, the lipid phase changes from gel to liquid state. The appropriate quantity of propylene glycol is heated to a desired, or to a maximum, transition temperature of the lipid blend. For example, in the present formulation, DSPC is the least soluble lipid, with a maximum transition temperature of 60 deg C. (highest among the lipids used). The lipid with less, or potentially, minimum solubility, is added first to the hot propylene glycol and the beaker is placed in a bath sonicator for gentle mixing. Sequentially, add the lipids to the beaker in the bath sonicator. Simultaneously, prepare a mixture of glycerol and buffer solution and heat it to the desired, or potentially, maximum transition temperature. Once the propylene glycol-lipid solution is translucent (free of white clumps) in the sonicator, mix it with the glycerol-buffer solution. The resultant solution is mixed on a magnetic platform with a temperature-controlled water bath. The temperature of the water bath preferably does not exceed the desired, or potentially, maximum transition temperature of the lipid by 5° C. The increase in temperature adversely affects the membrane rigidity. The lipid solution comprises of 15% propylene glycol, 5% glycerol and 80% PBS buffer by volume. Depending on the main lipid, the quantity of propylene glycol can be increased. For example, if the main lipid is DPPC, the lipid dissolution can be achieved even at 10% propylene glycol solution.

The mixing of lipid solution at the desired temperature may be done for one hour or longer. Afterwards, the lipid solution may be brought to room temperature by removing it from the bath. The solution may be stored at 4° C. for further use.

A homogenizer also can be used to mix the lipid-propylene glycol-Glycerol-buffer solution mixture. The homogenizer can be operated, for example, at 3000 rpm. The homogenization is preferably conducted for 1 hour or more with the temperature maintained above the desired, or potentially maximum, transition temperature of the lipids.

The prepared lipid solution was filtered to remove dust particles, undissolved lipid clumps, etc. Hydrophilic syringe filters were used for this process. The filters were soaked in the same temperature bath prior to their use. 2-micron, 0.8-micron and 0.45-micron filter were used in sequence for filtering the lipid solution.

TABLE 3
Lipids in the droplet shell
Total lipid concentration: 5 mg/ml

| | | | Molecular |
|---|---|---|---|
| | Molar ratio | CAS no | weight (gm) |
| DSPC | 60 | 816-94-4 | 790.15 |
| PEG40 Stearate | 35 | 9004-99-3 | 328.537 |
| DSPE-MPEG-2000 | 4 | 474922-77-5 | 2820 |
| DSPE-PEG-2000-BIOTIN | 1 | 385437-57-0 | 3070 |

TABLE 4
Mass (mg) of individual lipids for a given volume of lipid solution (ml)

| | | PEG40 | DSPE- | DSPE-PEG- |
|---|---|---|---|---|
| V (solution) | DSPC | Stearate | MPEG-2000 | 2000-BIOTIN |
| (ml) | (mg) | (mg) | (mg) | (mg) |
| 10 | 32.3 | 8.1 | 8.0 | 5.5 |
| 20 | 64.6 | 16.1 | 15.9 | 10.9 |
| 30 | 96.9 | 24.2 | 23.9 | 16.4 |
| 40 | 129.2 | 32.3 | 31.8 | 21.8 |
| 50 | 161.4 | 40.3 | 39.8 | 27.3 |
| 60 | 193.7 | 48.4 | 47.7 | 32.7 |
| 70 | 226.0 | 56.5 | 55.7 | 38.2 |
| 80 | 258.3 | 64.5 | 63.6 | 43.6 |
| 90 | 290.6 | 72.6 | 71.6 | 49.1 |
| 100 | 322.9 | 80.7 | 79.5 | 54.6 |
| 110 | 355.2 | 88.8 | 87.5 | 60.0 |
| 120 | 387.5 | 96.8 | 95.4 | 65.5 |
| 200 | 645.8 | 161.4 | 159.1 | 109.1 |
| 300 | 968.6 | 242.1 | 238.6 | 163.7 |
| 1000 | 3228.8 | 806.9 | 795.4 | 545.5 |
| 12000 | 38745.6 | 9682.3 | 9544.6 | 6546.3 |

Many formulations of the lipid coating/shell were developed. In some formulations DSPE-MPEG-2000 was replaced by DSPE-MPEG-5000 and correspondingly biotinylated lipid was changed to DSPE-PEG-5000-Biotin. DSPC also can be replaced by DPPC. The ratio of the biotinylated lipid was also varied to check its effect on the binding with the cells. In one of the formulations a part of DSPC was biotinylated and also a spacer was introduced to have better binding between biotin and the cell. The pegylated lipids were introduced to provide steric stability to the droplet. An emulsion stabilizer/cosurfactant like PEG40 Stearate was also used. All the lipids used here are biocompatible and, in the past, they have been used in various FDA approved liposome-based drugs.

Above mentioned formulation (Table 3) is based on biotin-Neutravidin non-covalent conjugation. Some lipid formulations were developed in which Neutravidin was directly conjugated to the droplet surface. In such formulations, the DSPE-PEG-2000-Biotin was replaced with DSPE-PEG-2000-Maleimide and the droplets manufactured after homogenization have Maleimide for further conjugation. The Maleimide containing droplets are conjugated with thiolated Neutravidin to generate a stable thioether bond with neutravidin on the surface. Although not preferable, the reaction between Maleimide and thiolated Neutravidin may also be done during the lipid preparation stage. With this approach, the high shear used for droplet preparation may denature the neutravidin. A pH˜7 should be maintained to avoid hydrolysis of lipid Maleimide. Conjugation should be done in atmosphere of Nitrogen or Argon.

In another example, the DSPE-PEG-2000-Biotin was replaced with DSPE-PEG-2000-Desthiobiotin in the lipid formulation. This modification was performed to make the droplets elutable at the end of positive selection process.

In another example, the Neutravidin was replaced with Streptavidin in the droplet manufacturing. It was observed from well plate experiments that desthiobiotin droplets eluted faster when they were non-covalently linked to Streptavidin. This modification may significantly reduce the elution time and may increase the elution efficiency.

In another example, a cationic lipid such as DOTAP, DOTMA may be included in the lipid formulation, to non-covalently attach it to a biotinylated ss-DNA or ds-DNA to have a droplet with DNA or RNA modification. The DNA modification in the droplet may be used for elution purpose by using a strand displacer or by benzonase.

In another example, DSPE-PEG-2000-Biotin may be replaced with DSPE-PEG-2000-Maleimide and the Maleimide lipid may be conjugated with a thiolated DNA strand. This may result in a lipid with a covalent DNA modification. The droplets can be prepared afterwards by the preferred mixing method.

In another example, a cationic lipid such as DOTAP, DOTMA may be included in the lipid formulation, which may permit the PFH droplets to be used in applications that call for transfection of DNA to cell membrane.

In another example, the core of the droplet may be modified by having a mixture of different perflurocarbons (PFCs). Mixing a higher molecular weight PFC increases the shelf life of the PFC emulsion (Davis & Wotton, 1989). For example, Perfluorohexane (PFH) may be mixed with Perfluorodecalin (PFD) in 90:10 ratio and the mixture may be used as the droplet core.

In another example, the emulsion stabilizer such as PEG40 Stearate can be used in larger quantity to increase the viscosity of the final droplet solution. High viscosity of the solution reduces the motion of droplets in the solution and in turn reduces the rate of coalescence. This modification may have a significant positive effect on the shelf life of the PFH droplets.

Preparation of Small Size Droplet

in some examples, PFH, PFOB, and PFD were used for droplet manufacturing. The detailed results are presented here for droplets made of PFH. The lipid solution is mixed with PFH liquid in a narrow vessel. The denser PFH liquid tends to fall to the bottom of the container and the lipid solution tends to rise to the top. Both the lipid solution and the PFH liquid are transparent, but a sharp interface can be seen. To make small size droplets, the amount of PFH liquid in the container is preferably limited, e.g., to a minimum. As the ratio of PFH volume to lipid solution volume increases, the size of the droplets increases until a plateau is reached for a given sonication power, for example. The PFH liquid is low strength, as it has a low surface tension value. Therefore, the sonication amplitude is selected appropriately to overcome the surface tension value. The input of the ultrasonic acoustic wave can be provided in a pulsed mode. In some examples, continuous mode of the ultrasonic acoustic wave may be avoided. The tip of the horn may be placed at the interface of PFH and lipid solution. The placement of the tip at the interface influences, and in some examples is critical to, the consistency of the size distribution of droplets. The size distribution may change if a different size container is used for sonication. To avoid formation of bubbles/foam the horn is placed sufficiently inside the solution. In this example, the aim is to prepare a droplet solution and not a bubble solution, so the narrow vessel is submerged in a transparent low temperature bath. The transparent low temperature bath is made by making a supersaturated solution of salt and then storing the salt solution in the freezer at −20 deg C. The tip of the horn sonicator used in these experiments has a diameter of 0.5 inch.

Small Droplets Protocol
1. The lipid-PFH solution is sonicated.
   a. Horn sonicator: 0.5 inch probe and 750 Watt max power.
2. In a cuvette, pour 2 ml of Perfluorohexane.
3. Pour 4 ml of lipid solution into the same cuvette (shake the stock of lipid solution very gently before using it).
4. Place the tip of the sonicator at the interface of perfluorohexane and lipid solution.
5. Use the transparent low temperature bath for cooling the sample holder.
6. Sonication parameters (PFH): 13% Amplitude, 2 Sec On: 8 Sec Off, Total process time 10 Secs.
7. Centrifugation (Use a buffer solution with 2% BSA).

Large Droplets Protocol
1. In a 15 ml centrifuge tube, mix 4 ml of PFH and 6 ml of lipid solution.
2. After a gentle mixing pour the solution in a 30 ml beaker.
3. Use the homogenizer (IKA T25 ULTRA TURRAX) at 10,000 rpm for 45 secs.
4. Centrifugation (Use a buffer solution with 2% BSA).

The size measurement of the droplets was performed using a Beckman Coulter Multisizer. For small droplets, an aperture of 20 microns was used, whereas for the larger droplets a 50 micron aperture was used. The size distribution for small droplets has a concentration of 24 Billion/ml of particles with a diameter greater than about 0.9 μm and a volume percentage of about 50% by volume. The size distribution for large droplets has a concentration of 1.22 billion/ml of particles with a diameter greater than about 2 μm and a volume percentage of about 50% by volume.

In the above examples, a PFC liquid and a lipid solution are combined to make a liquid core with a lipid shell. The PFC liquid is dispersed in another solution to form droplets. An emulsifier may be added to the solution, to prevent the droplets from coalescing. In some embodiments, phospholipids are used as the emulsifier/surfactant. A PFC liquid is dispersed by different methods depending upon the size of droplets desired for the application. To create small nanometer-sized droplets, ultrasonic agitation may be used. To create larger droplets, a vial shaker may be used to agitate the liquid mixture.

In some embodiments, a lipid solution consists of several different lipid materials in solution. The procured lipids are stored in a freezer at about −20° C. At this temperature, the lipids are in a solid state. The lipids may be taken out of the freezer and left at room temperature for about 20 minutes before use. This is done to bring the lipids to gel state. Since lipids generally do not dissolve in water, propylene glycol may be used to dissolve them. It is preferable to not dissolve all the lipids at once in the propylene glycol, as putting all the lipids at the same time may result in formation of white clumps in the solution. The solubility of each lipid material was compared and the lipid material with maximum solubility was dissolved first in the propylene glycol, followed by the next most soluble lipid material, and so on. Since the solubility of the lipids are a function of temperature of the solution, the solution was maintained at a temperature above the transition temperature of the lipids. Table 5 is an example of a lipid composition.

TABLE 5
Lipids

| Total lipid | 1 mg/ml | | | |
|---|---|---|---|---|
| concentration | | | | |
| | | | Avanti | |
| | Mol wt | Molar | catalog | No of |
| | (gm) | ratio | information | carbons |
| DPPA | 670.87 | 11 | | 16 |
| DPPC | 734.04 | 82 | | 16 |
| DPPE-PEG-5000 | 5744 | 0 | 880200 | 16 |
| DSPE-PEG-2000 | 2805.49 | 0 | 880120 | 18 |
| DSPE-PEG-2000- | 3070 | 0 | 880129 | 18 |
| BIOTIN | | | | |
| DSPE-PEG-5000- | 5670 | 7 | | 18 |
| BIOTIN | | | | |

| V (stock lipid | DPPA | DPPC | DSPE-PEG-5000- |
|---|---|---|---|
| volume), mL | (mg) | (mg) | BIOTIN (mg) |
| 10 | 0.69 | 5.61 | 3.70 |
| 20 | 1.38 | 11.22 | 7.40 |
| 30 | 2.06 | 16.84 | 11.10 |
| 40 | 2.75 | 22.45 | 14.80 |
| 50 | 3.44 | 28.06 | 18.50 |
| 60 | 4.13 | 33.67 | 22.20 |
| 70 | 4.82 | 39.28 | 25.90 |
| 80 | 5.50 | 44.89 | 29.60 |
| 90 | 6.19 | 50.51 | 33.30 |
| 100 | 6.88 | 56.12 | 37.00 |
| 110 | 7.57 | 61.73 | 40.70 |
| 120 | 8.26 | 67.34 | 44.40 |

An example process for creating a lipid solution is as follows. The propylene glycol is heated to the maximum transition temperature of the lipid blend for mixing. The lipid material with maximum solubility is added to the heated propylene glycol. The lipid material and propylene glycol are mixed in a bath sonicator. Sequentially, lipids of lower solubility are added into the propylene glycol mixture while in the bath sonicator.

A mixture of glycerol and buffer solution may be prepared simultaneously. The glycerol and buffer solution is heated to the maximum transition temperature. Once the lipid-propylene glycol solution is translucent (free of white clumps) in the sonicator, the lipid-glycol solution is mixed with the glycerol-buffer solution. The resulting mixture is homogenized with a homogenizer operating at 3000 rpm. The homogenization is performed for about one hour. During the homogenization process, the temperature is maintained at the maximum transition temperature of the lipids.

The prepared lipid solution is filtered to remove any possible contaminants such as dust, undissolved lipid clumps, etc. The filtering process may be performed with a hydrophilic syringe filer. The filters are soaked in the same temperature batch prior to use. In some embodiments, a 2.0 micron filter is used. In other embodiments, a 0.8 micron filter is used. In yet other embodiments, a 0.45 micron filter is used. In some embodiments, a combination of filters may be used.

The lipid solution is mixed with the PFC liquid in a narrow vessel to create core-shell particles. The PFC liquid is placed into a vessel and the lipid solution is poured on top. To make smaller sized droplets, the amount of PFC liquid in the vessel is reduced. As the ratio of PFC liquid volume to lipid solution volume increases, the size of the formed droplet increases until it reaches a plateau for a given sonication power. The PFC liquids are low strength as they have low surface tension values. Therefore, the sonication amplitude should be selected appropriately and the input of ultrasonic waves should be done in a pulsed mode rather than in a continuous mode. The tip of a horn sonicator assembly should be placed at the interface of two liquid solutions. To avoid formation of bubbles/foam the horn should be sufficiently inside the solution. Here, the aim is to prepare a droplet solution, so the narrow vessel is submerged in a transparent low temperature bath. The transparent low temperature bath is made, for example, by making a supersaturated solution of salt and then storing the salt solution in the freezer at −20° C. The sonication produces smaller beads.

In one example, the lipid solution may comprise about 1 mL propylene glycol+1 mL glycerol+8 mL buffer solution+lipid blend of 10 mg. 9 mL of the lipid solution may be combined with about 1 mL of PFC solution. The Lipid-PFC solution may be sonicated. For a 0.5 inch probe and 750 watt sonicator, a PFC solution utilizing 30% PFP is sonicated for about 3 seconds on and about 10 seconds off until a total sonication time of about 15 seconds is reached. A PFC solution utilizing 40% PFH is sonicated for about 3 seconds on and about 10 seconds off until a total sonication time of about 15 seconds is reached. A PFC solution utilizing 50% PFOB is sonicated for about 3 seconds on and about 10 seconds off until a total sonication time of about 15 seconds is reached. The sonication produces a droplet solution.

To prepare larger sized droplets, the quantity of PFC liquid is increased and the power input of the sonicator is reduced drastically. In another non-limiting exemplary embodiment, 500 microliters of PFC and 2 mL of lipid solution may be placed in a 3 mL vial. The vial may then be shaken in a vial mixer at 4800 rpm for 30 seconds. The prepared droplet suspension may have some microbubbles. In cases where microbubbles are present, the solution may be centrifuged.

### EXAMPLES

### Example 1 Perfluorohexane Droplets Used for Negative Selection

Small droplet manufacturing protocol (Sonication): The lipid-PFH solution is sonicated using a horn sonicator (0.5 inch probe, 750 Watt max power). In a cuvette, pour 2 ml of Perfluorohexane and 4 ml of lipid solution. The tip of the sonicator was placed at the interface of perfluorohexane and lipid solution. A transparent low temperature bath was used for cooling the sample holder. A sonication amplitude of 13% was used and it was operated in pulsed mode. A 2 sec on and 8 sec off pulse wave was used for 5 times to have an effective sonication time of 10 secs. The sonication produces highly polydispersed population, so multiple centrifugation wash was performed to get rid of very small droplets. A 2% BSA-DPBS buffer was used for washing and dilution during the centrifugation. FIG. 4 shows the final size distribution after centrifugation steps. Beckmann coulter counter (Multisizer) was used for size measurement of the sample.

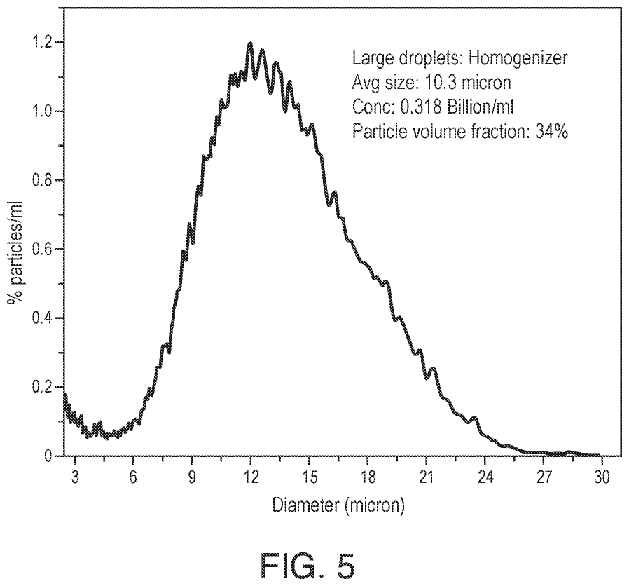

Large Droplets Protocol (Homogenization): 4 ml of PFH and 6 ml of lipid solution was poured in a 30 ml beaker and homogenization was performed. IKA T25 ULTRA TURRAX homogenizer was used at 10,000 rpm for 45 secs. After homogenization multiple centrifugation wash were performed to achieve the desired size. FIG. 5 shows the final size distribution after centrifugation steps.

### Example 2 Perfluorohexane Droplets Used for Positive Selection

Objective: isolate CD4+ and CD8+ T cells from a Leukopak.

Droplet manufacturing: The PFH droplets were prepared in large volume by using a homogenizer. The process was modified to produce the droplets on industrial scale. 480 ml of lipid solution was mixed with 320 ml of perfluorohexane liquid in a beaker at 25000 rpm for 4 minutes. The beaker was jacketed with ice cold water. Homogenization results in a very polydisperse population and multiple steps of centrifugation were done to achieve the desired population. The aim was to get the mean size between 5-8 micron diameter.

Centrifugation: The aim is to get rid of very small droplets as they may not be held in the column at the desired acoustic power. A large centrifugation cup of 500 ml was used for the centrifugation purpose. All the speeds used in this study were calculated for a centrifuge machine with Rmin=100 mm and Rmax=205 mm. Following are the details of each of the centrifugation step.

C1: 300 ml of buffer was filled in the centrifuge cup and afterwards 200 ml of initial droplet solution was gently poured in the cup. Centrifugation was performed at 500 rpm for 3 mins.

C2: The supernatant from previous step was removed and the pellet was collected in a beaker. The cup is cleaned and filled again with 300 ml of buffer and the pellet (reformulated to 200 ml) was poured into it gently. Centrifugation was performed at 500 rpm for 3 mins.

C3: The supernatant from previous step was removed and the pellet was collected in a beaker. The cup is cleaned and filled again with 300 ml of buffer and the pellet (reformulated to 200 ml) was poured into it gently. Centrifugation was performed at 450 rpm for 3 mins.

C4: The supernatant from previous step was removed and the pellet was collected in a beaker. The cup is cleaned and filled again with 300 ml of buffer with 2% BSA and the pellet (reformulated to 200 ml) was poured into it gently. Centrifugation was performed at 450 rpm for 2 mins.

C5: The pellet was collected after 4 steps of centrifugation. The droplet solution was incubated with a sufficient quantity of neutravidin at 4.0 for 1 hour. The amount of neutravidin depends on the mean size and the concentration of droplet solution after 4 steps of centrifugation.

C6: The incubated neutravidin droplet solution is poured in a 500 ml centrifugation cup filled with 2% BSA solution to wash the unbounded neutravidin. Centrifugation was performed at 450 rpm for 3 mins (BSA sol 300 ml+Incubated Droplet sol 200 ml).

C7: The supernatant from previous step is removed and reformulated to 200 ml and poured in a centrifugation cup already filled with 2% BSA solution (300 ml). The centrifugation was performed at 450 rpm for 2 mins.

C8: The supernatant is removed from the centrifugation cup and the droplet sample is collected in a vial for size distribution measurements

As another example, the exterior layer of the particle may be useful for causing biological interaction/reaction of the particle. For example, the exterior layer may permit the particle to be used for affinity binding.

Calculation of quantity of Neutravidin for a given size and concentration: The PFC droplets are conjugated with Neutravidin to make them ready for binding cells that are biofunctionalized with biotinylated antibodies. Once the droplets are synthesized and centrifuged to get the desired size population, they are mixed with a sufficient quantity of Neutravidin solution. The amount of Neutravidin depends on the quantity of the biotinylated lipid, DSPE-PEG-2000-Biotin or DSPE-PEG-2000-Desthiobiotin, that is in the shell. Neutravidin should be added in excess quantity, so that it covers all the biotin sites on the droplet. If the droplet solution is not saturated with neutravidin, then it may lead to cross-linking between the droplets. Here we present the calculation of Neutravidin for a given droplet size and concentration.

The calculation assumes that all the biotin is available for binding and there is no cross-linking of droplets. The mean size of the droplet population is considered for the neutravidin calculation. Based on the molar ratio and surface area of the molecules for each type of lipid, the number of available biotin sites can be calculated on a droplet of given size. From the number of biotin sites, the total mass of neutravidin can be calculated for a given droplet population. Table 6 shows the topological planar area of a single molecule of all the lipids. Table 7 and Table 8 show the neutravidin calculation for 1 ml of small and large droplets, respectively. The droplets are incubated with neutravidin solution at 4° C. for 30 minutes. After incubation the droplet-neutravidin solution should be washed twice to remove any unbounded Neutravidin. Any unbounded Neutravidin in the solution will block the binding sites on cells (biofunctionalized with biotinylated antibodies) during incubation.

TABLE 6
Topological planar area of single lipid molecule.

| | Area/molecule nm² | molar ratio |
|---|---|---|
| DSPC | 1.11 | 60 |
| PEG40 Stearate | 0.465 | 35 |
| DSPE-PEG-2000-BIOTIN | 1.84 | 1 |
| DSPE-MPEG-2000 | 1.59 | 4 |

TABLE 7
Example: Neutravidin used for 1 ml of small droplet.

| | | |
|---|---|---|
| Mean diameter (nm) | 1500 | |
| Number of biotin sites on 1 droplet | 19394 | |
| | Daltons | mg |
| Weight of 1 molecule of neutravidin | 60000 | 9.96318E−17 |

| | |
|---|---|
| Total weight used for 1 droplet | 1.9322E−12 |
| Droplet count/ml | 2.40E+10 |
| Total weight (mg) | 4.64E−02 |
| Factor of safety | 20 |
| Net weight used for 1 ml small droplets (mg) | 0.93 |

TABLE 8
Example: Neutravidin used for 1 ml of large droplet.

| | | |
|---|---|---|
| Mean diameter (nm) | 9000 | |
| Number of biotin sites on 1 droplet | 698160 | |
| | Daltons | mg |
| Weight of 1 molecule of neutravidin | 60000 | 9.96318E−17 |

| | |
|---|---|
| Total weight used for 1 droplet | 1.9322E−12 |
| Droplet count/ml | 1.22E+09 |
| Total weight (mg) | 8.49E−02 |
| Factor of safety | 20 |
| Net weight used for 1 ml small droplets | 1.70 |
| (mg) | |

### Example 3 Perfluorohexane Droplets Used for Positive Selection and Suitable for Elution

Objective: isolate CD4+ and CD8+ T cells from a Leukopak and perform elution of droplets from cells.

Droplet manufacturing: The PFH droplets use biotin neutravidin bond to target the cell. As the aim is not only to isolate the cell but to finally elute the droplets, so a modified form of biotin was used in the lipid preparation. The DSPE-PEG 2000-Biotin in Example 2 was replaced with DSPE-PEG-2000-Desthiobiotin to achieve the elution. The desthiobiotin molecule has just one ring compared to two rings in the regular biotin molecule and it has less affinity for neutravidin compared to regular biotin droplets (Hirsch et al., 2002). After manufacturing the droplets with desthiobiotin lipids the droplets are incubated with Neutravidin. To achieve elution, the desthiobitin droplet cell complex was incubated in a 50 mM biotin buffer solution for 2 hours at 37° C. As free biotin present in the buffer has more affinity for Neutravidin/Streptavidin, it displaces the desthiobiotin droplets linked non-covalently to Neutravidin. The other steps in the droplet manufacturing were performed as provided in Example 2. The size distribution of the desthiobiotin droplets is similar to the regular biotin droplets manufactured in Example 2.

Measurement of Biotin-Binding Capacity of Droplets Via Flow Cytometry

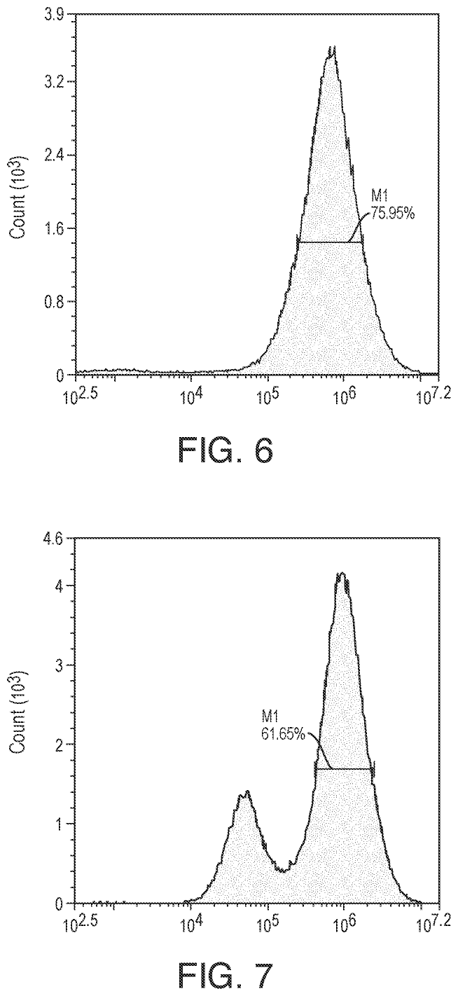

The droplets used for acoustic affinity cell selection (AACS) are coated with NeutrAvidin, a deglycosylated form of streptavidin. NeutrAvidin has a very high affinity (KD=10-15) for biotin. The amount of biotin that the NeutrAvidin on the droplets can bind per unit surface area (biotin binding capacity) is calculated to improve successful binding in the AACS column. Fluorescent biotin (Biotin-APC conjugate) is labelled to the droplets with Neutravidin. The fluorescence is calibrated and biotin binding is measured using flow cytometry. FIG. 6 shows the biotin binding capacity for a regular biotin droplet. FIG. 7 shows the binding capacity for desthiobiotin droplets. Both types of droplets have similar biotin binding capacity. The biotin binding capacity varies from batch to batch between 6-12 pmol biotin/cm2 of the droplet surface. FIGS. 6 and 7 show the count on the y-axis and mean fluorescence intensity on the x-axis. The desthiobiotin droplets binding capacity plot has two peaks compared to a single plot in regular biotin droplets. The second peak in the desthiobiotin droplet plot may be attributed to use of Biotin-APC used for the measurement. The biotin APC may have displaced the desthiobiotin droplets from the neutravidin and the second peak may be signal from such clusters

In a cell selection example, the droplets are used to perform a cell isolation test. For this test, the droplets may be manufactured with desthiobiotin. The cells (target and non-target) are incubated with anti-CD4 biotin and anti-CD8 biotin antibodies and the droplets are loaded into an acoustic separation column. Once the column is loaded with the droplets, a 1% BSA-PBS buffer is flushed through the column and the acoustics are switched on. In few minutes a zone of droplet suspension is formed below the edge of the generated acoustic field. After a stable edge is formed, the cell suspension is loaded in the column and subsequently the cells with antibodies attach to the droplets. The flow of the flush buffer is continued, and it flushes out free, unbound cells. After a time interval where the free cell leaves the column and elution process can be implemented. The elution process may include supplying a biotin elution buffer to the column to elute the desthiobiotin droplets from the cells. The flow may be recirculated, during which time the temperature of the column may be elevated to 37° C. Higher temperature and shear may accelerate the elution. During the elution phase the acoustics are switched off. After 1 hour of recirculation, the acoustics are switched on and a flush buffer is used to separate the eluted cells from the droplets. As the cells are not as acoustically responsive as the PFH droplets, they are not retained in the column by the acoustics, whereas the PFH droplets are retained below the acoustic edge. This overall process yields a high elution efficiency.

In other examples, positive and negative selection of cells were performed using perfluorohexane (PFH) droplets and high purity and high recovery of target cells were achieved. For the sake of comparison, negative selection of TCR cells were performed and PFH droplets and Promega beads were used. The purity and recovery because of PFH droplets is significantly higher than the Promega bead. Both perfluorocarbon and phospholipid are biocompatible and they have been used in the past in various drugs. The perfluorohexane droplet not only facilitate cell separation but also, they can be modified to achieve elution. The biotin present on the droplets can be modified to desthiobiotin and an elution buffer containing free biotin molecule can be used for eluting the PFH droplet from cell complex.

The PFH droplet yields higher purity and recovery of the target cells. Both PFH and phospholipids are biocompatible. They have a proven track record of being used in FDA approved drugs.

The Commercially available platforms are limited in their scale and ability to handle complex input such as Leukopak, most of them are designed to use PBMC as their input. The PFH droplet facilitates a continuous process to isolate the cell. To scale up the process, the volume of column, acoustics input, flow rate and PFH droplet quantity can be changed accordingly. We have demonstrated to handle few millions of cells to multibillions of cells in different acoustic column volumes and chamber.

The overall cell isolation using PFH droplets takes less than 4 hours, which is significantly below the time taken by nearest competitor. The commercially available Miltenyi products cannot operate at higher flow rate in a single column, because of the design limitation of the column. They are limited by the width of the channels between spheroids (˜20 times size of lymphocytes). The high intensity magnetic field is present near the boundary of the spheroids and in most part of channels it is of low magnitude. Increasing the width may compromise the capturing of cells attached to the magnetic bead. The PFH droplet are not constrained by any such operational flow rate restrictions.

As the size of Miltenyi beads is 100-200 nm, so they may internalize to the cells during the process. The PFH beads used here have a mean size between 5-7 μm, so the chance of internalization is minimal.

The fluorinated droplets are conjugated with Neutravidin to make them ready for binding cells that are biofunctionalized with biotinylated antibodies. Once the droplets are synthesized and centrifuged to get the desired size population, they are mixed with the desired quantity of Neutravidin solution. The amount of Neutravidin depends on the quantity of the biotinylated lipid, DSPE-PEG-2000-Biotin, that is in the shell. Neutravidin can be added in excess quantity, so that it covers all the biotin sites on the droplet. If the droplet solution is not saturated with neutravidin, then it may lead to cross-linking between the droplets. Here we present the calculation of Neutravidin for a given droplet size and concentration.

The Leukopak was incubated with an appropriate amount of antibody for 30 mins and was loaded to the acoustic affinity column. The binding between cell-antibody and droplet occurs in the column. The non-target cells pass through the acoustic chamber as they are not acoustically responsive whereas the target cell-droplet complex is held back in the column. The column was flushed with buffer to remove the non-target cells from column. After certain time (depending on the cell quantity, column volume), the flushing process was stopped and elution of target cells from the droplet was initiated with a corresponding elution technique based on the kind of targeting mechanism. The targeting mechanism could be based on antibody, aptamer or antibody-oligo conjugates. Purity and recovery of the target cells were calculated using equation 1 and 2. It is to be noted that the purity and recovery presented in the subsequent section is based on the conservation of cell count and not based on the elution.

$$Retrention = 1 - \frac{{V}_{FL} CD 45 {count}_{FL} Target cell {\%}_{FL}}{{V}_{initial} CD 45 {count}_{initial} Target cell {\%}_{FL}}$$

$$Purity = \frac{\begin{matrix}{V}_{initial} CD 45 {count}_{initial} Target cell {\%}_{initial} - \\ {V}_{FL} CD 45 {count}_{FL} Target cell {\%}_{FL}\end{matrix}}{{V}_{initial} CD 45 {count}_{initial} - {V}_{FL} CD 45 {count}_{FL}}$$

Counting Method: Blood Analyzer Counts Multiplied by Flow Cytometry Percentages.

| | | | | Flow | |
|---|---|---|---|---|---|
| | | Antibody | Cell | Rate | Droplet |
| Sample | Cell Type | Type | Concentration | ml/min | Conc. |
| C | Apheresis pdt | CD4 & CD8 | 1x | 1 | 15% |
| B | Apheresis pdt | CD4 & CD8 | 1x | 1 | 15% |
| A | Apheresis pdt | CD4 & CD8 | 1x | 1 | 15% |
| D | Apheresis pdt | CD4 & CD8 | 1x | 1 | 15% |
| E | Apheresis pdt | CD4 & CD8 | 1x | 1 | 15% |

| | | | | | Total |
|---|---|---|---|---|---|
| | | | | | Target |
| | | | | | Cell |
| Sample | Power | Column Type | Temperature | Wash | Number |
| C | 0.5 W | 5 ml | RTP | yes | 300M |
| B | 0.5 W | 5 ml | RTP | yes | 600M |
| A | 0.5 W | 5 ml | RTP | yes | 1200M |
| D | 0.5 W | 5 ml | RTP | yes | 1800M |
| E | 0.5 W | 5 ml | RTP | yes | 2400M |

Antibody: 1 Kd amount for CD4 antibodies per million target cells and 1 Kd amount for CD8 antibodies per million target cells.

% Uncertainty in Retention Measurements.

| Column | Target Input | % Uncertainty |
|---|---|---|
| C | 300M | 4 |
| B | 1600M | 6 |
| A | 1200M | 3 |
| D | 1800M | 4 |
| E | 2400M | 4 |

% Uncertainty in Target Purity Measurements.

| Column | Target Input | % Uncertainty |
|---|---|---|
| C | 300M | 0.84 |
| B | 600M | 0.99 |
| A | 1200M | 0.92 |
| D | 1800M | 1.92 |
| E | 2400M | 1.76 |

Results: The cell isolation was performed in acoustic affinity fluidized bed column. The results reported below were collected from experiments on three different day, each day testing three different types of particles under the same conditions. The particles tested were small droplets, large droplets, and Promega beads. Each day the droplets were loaded into a 5 mL column at a concentration of approximately 20% solids. The columns were cooled during the experiment, which consisted of a single pass of and initial 10 mL sample of 100M total cells, and a 30 mL buffer flush. Flow rates were 1 mL/min for all columns, and power was 1 W for columns containing the small droplets and 0.6 W for columns containing large droplets or Promega beads.

For each experiment, purity and recovery of TCR-cells were calculated using the following formulas:

$$Purity = \frac{{TCR}^{-} cells out}{{TCR}^{+} cells out + {TCR}^{-} cells out}$$

$$Recovery = \frac{{TCR}^{-} cells out}{Initial {TCR}^{-} cell count}$$

Positive and negative selection of cells may also be performed using various particles. For instance, the negative selection of TCR positive T cells is a process where functionalized particles bind with TCR positive T cells such that the TCR positive T cell is removed from the system. TCR positive T cells are deleterious to processes such as chimeric antigen receptor T cell therapies (CAR-T).

A positive selection process may also be utilized for specific cells where modified T-cells are selected by appropriately functionalized particles such that they are culled from a cell culture to then subsequently be utilized in a cellular therapy.

The results reported below were collected from experiments on three different days, each day testing three different types of particles under the same operating conditions. The particles tested were small droplets, large droplets, and Promega beads. Each day, the droplets were loaded into a 5 mL column at a concentration of approximately 20% volume. The columns were cooled during the experiments, which consisted of a single pass with an initial 10 mL sample of 100M total cells, and a 30 mL buffer flush. Flow rates were 1 mL/min for all columns, and power was 1 W for columns containing the small droplets and 0.6 W for columns containing large droplets or Promega beads. One small difference between the three days was the use of smaller diameter tubing on the third day. This change significantly reduced the holdup volume of the system and allowed for analysis of the initial outflow sample for the third day since the sample was less diluted. For the purposes of this report, however, the outflow sample will not be used in the analysis so that analysis is consistent between all days.

For each experiment, three metrics are calculated and used to compare the types of particles to one another. Total purity, recovery of TCR-cells, and TCR+ cell depletion efficiency are all calculated using the following formulas:

$$Total Purity :$$

$$Purity = \frac{Sum of {TCR}^{-} cells out}{sum of {TCR}^{+} cells out}$$

$${TCR}^{-} cell Recovery :$$

$$Recovery = \frac{Sum of {TCR}^{-} cells out}{Initial {TCR}^{-} cell count}$$

$${TCR}^{+} Cell Depletion Efficiency :$$

$$Depletion Efficiency = 1 - ( \frac{Sum of {TCR}^{+} cells out}{Inital {TCR}^{+} cell count * Recovery} )$$

Test 1

TABLE 9 a.
Test 1 Results.

| Test | Purity | Recovery | Depletion Efficiency |
|---|---|---|---|
| Small Droplets | 98.4% | 46.9% | 94.2% |
| Large Droplets | 95.7% | 55.6% | 87.8% |
| Promega Beads | 90.4% | 30.1% | 70.5% |

TABLE 9 b.
Test 1 Results.

| Particle | | | Total Viable | TCR− Cell | TCR+ Cell |
|---|---|---|---|---|---|
| Type | Fraction | Purity | Cell Count | Count | Count |
| Small | Feed | 78.3% | 9.32E+07 | 7.30E+07 | 2.02E+07 |
| Droplets | Flush 1 | 97.9% | 1.54E+07 | 1.51E+07 | 3.29E+05 |
| | Flush 2 | 99.1% | 1.45E+07 | 1.43E+07 | 1.27E+05 |
| | Flush 3 | 98.1% | 4.84E+06 | 4.75E+06 | 9.06E+04 |
| Large | Feed | 73.1% | 1.01E+08 | 7.38E+07 | 2.71E+07 |
| Droplets | Flush 1 | 97.8% | 1.71E+07 | 1.67E+07 | 3.78E+05 |
| | Flush 2 | 95.4% | 1.83E+07 | 1.75E+07 | 8.36E+05 |
| | Flush 3 | 91.5% | 7.41E+06 | 6.78E+06 | 6.31E+05 |
| Promega | Feed | 73.4% | 9.75E+07 | 7.16E+07 | 2.59E+07 |
| Beads | Flush 1 | 85.3% | 6.07E+06 | 5.18E+06 | 8.92E+05 |
| | Flush 2 | 91.1% | 1.31E+07 | 1.20E+07 | 1.17E+06 |
| | Flush 3 | 94.8% | 4.64E+06 | 4.40E+06 | 2.40E+05 |

Test 2

TABLE 10 a.
Test 2 Results.

| Test | Purity | Recovery | Depletion Efficiency |
|---|---|---|---|
| Small Droplets | 98.2% | 21.5% | 93.0% |
| Large Droplets | 97.0% | 20.6% | 88.9% |
| Promega Beads | 83.5% | 13.6% | 49.5% |

TABLE 10 b.
Test 2 Results.

| Particle | | | Total Viable | TCR− Cell | TCR+ Cell |
|---|---|---|---|---|---|
| Type | Fraction | Purity | Cell Count | Count | Count |
| Small | Feed | 71.3% | 1.09E+08 | 7.77E+07 | 3.13E+07 |
| Droplets | Flush 1 | 97.7% | 1.11E+06 | 1.09E+06 | 2.54E+04 |
| | Flush 2 | 99.2% | 1.00E+07 | 9.92E+06 | 8.20E+04 |
| | Flush 3 | 96.6% | 5.85E+06 | 5.65E+06 | 2.02E+05 |
| Large | Feed | 73.0% | 1.08E+08 | 7.89E+07 | 2.92E+07 |
| Droplets | Flush 1 | 98.7% | 1.36E+06 | 1.34E+06 | 1.79E+04 |
| | Flush 2 | 98.9% | 9.33E+06 | 9.22E+06 | 1.05E+05 |
| | Flush 3 | 94.7% | 5.97E+06 | 5.65E+06 | 3.18E+05 |
| Promega | Feed | 70.4% | 1.06E+08 | 7.44E+07 | 3.13E+07 |
| Beads | Flush 1 | 76.6% | 1.79E+06 | 1.37E+06 | 4.20E+05 |
| | Flush 2 | 81.4% | 6.75E+06 | 5.50E+06 | 1.26E+06 |
| | Flush 3 | 90.8% | 3.55E+06 | 3.22E+06 | 3.25E+05 |

Test 3

TABLE 11 a.
Test 3 Results.

| | Purity | Recovery | Depletion Efficiency |
|---|---|---|---|
| Small Droplets | 99.4% | 31.2% | 98.5% |
| Large Droplets | 96.9% | 79.6% | 92.0% |
| Promega Beads | 86.5% | 44.4% | 59.9% |

TABLE 11 b.
Test 3 Results.

| | | | Total | | |
|---|---|---|---|---|---|
| Particle | | | Viable Cell | TCR− Cell | TCR+ Cell |
| Type | Fraction | Purity | Count | Count | Count |
| Small | Feed | 71.9% | 1.01E+08 | 7.17E+07 | 2.93E+07 |
| Droplets | Flush 1 | 99.1% | 1.03E+07 | 1.02E+07 | 1.03E+05 |
| | Flush 2 | 99.5% | 7.48E+06 | 7.41E+06 | 7.48E+04 |
| | Flush 3 | 99.8% | 4.96E+06 | 4.96E+06 | 0.00E+00 |
| Large | Feed | 72.4% | 1.05E+08 | 7.35E+07 | 3.15E+07 |
| Droplets | Flush 1 | 99.1% | 3.16E+07 | 3.12E+07 | 3.16E+05 |
| | Flush 2 | 97.4% | 2.10E+07 | 2.01E+07 | 8.39E+05 |
| | Flush 3 | 89.2% | 9.90E+06 | 8.71E+06 | 1.19E+06 |
| Promega | Feed | 73.2% | 1.02E+08 | 7.24E+07 | 2.96E+07 |
| Beads | Flush 1 | 83.6% | 2.49E+07 | 2.07E+07 | 4.24E+06 |
| | Flush 2 | 90.8% | 1.01E+07 | 9.08E+06 | 1.01E+06 |
| | Flush 3 | 94.9% | 3.37E+06 | 3.20E+06 | 1.68E+05 |

Example of binding and elution (Neutravidin with Desthiobiotin droplets): The Leukopak was incubated with an appropriate amount of antibody and after 30 mins was loaded to the acoustic affinity column. The binding between cell-antibody and droplet occurs in the column. The non-target cells pass through the acoustic chamber as they are not acoustically responsive, whereas the target cell-droplet complex is held back in the column. The column was flushed with buffer to remove the non-target cells from column. After certain time (depending on the cell quantity), the flushing process was stopped and elution of target cells from the droplet was initiated by flowing a 50 mM biotin buffer in the column. The biotin buffer was recirculated at higher flow rate to create high shear. Under high shear the desthiobiotin neutravidin interaction reduces significantly and this may reduce the overall elution time and may increase the elution efficiency. After the recirculation of biotin buffer for 1 hour, the flowrate is reduced, and an edge is formed at the boundary of the acoustics. The edge formation facilitates the flush out of the eluted CD4 and CD8 T cells whereas the naked droplets are held back. The binding was performed in a 50 ml column with acoustic chamber of size 1×1 inch. The binding between droplet and cell antibody complex was performed at room temperature and the flow rate was 12.5 ml/min. The column was loaded with 15% of droplets by volume. 35 ml of Leukopak was used and it yielded 2.4 billion T cells. The power was kept at 14 W for this flow rate, to avoid trapping of unwanted cells in the acoustic chamber. The elution mechanism was based on desthiobiotin droplets. The formula for purity and recovery changes if elution is taken into account.

Example of binding and elution (Streptavidin with Desthiobiotin droplets): The Leukopak was incubated with an appropriate amount of antibody and after 30 mins was loaded to the acoustic affinity column. The binding between cell-antibody and droplet occurs in the column. The non-target cells pass through the acoustic chamber as they are not acoustically responsive, whereas the target cell-droplet complex is held back in the column. The column was flushed with buffer to remove the non-target cells from column. After certain time (depending on the cell quantity), the flushing process was stopped and elution of target cells from the droplet was initiated by flowing a 100 mM biotin buffer in the column. The biotin buffer was recirculated using an oscillatory flow. The oscillatory flow enhances the mixing and may increase the diffusion of biotin to elution sites, thereby decreasing the elution time and elution efficiency. After the recirculation of biotin buffer for 1 hour, the flowrate is reduced, and an edge is formed at the boundary of the acoustics. The edge formation facilitates the flush out of the eluted CD4 and CD8 T cells whereas the naked droplets are held back. The binding was performed in a 50 ml column with acoustic chamber of size 1.5×1.5 inch. The binding between droplet and cell antibody complex was performed at room temperature and the flow rate was 20 ml/min. The column was loaded with 15% of droplets by volume. 35 ml of Leukopak was used and it yielded 4.45 billion T cells. The power was kept at 20 W for this flow rate, to avoid trapping of unwanted cells in the acoustic chamber. The elution mechanism was based on desthiobiotin droplets. The formula for purity and recovery changes if elution is taken into account.

In this example, Recovery=CD4 in elution product/CD4 in feed. Purity=(CD4+CD8)/CD45. Efficiency=CD4+CD8 in elution/CD4+CD8 in column. Recovery=CD4+CD8 in elution/CD4+CD8 in feed. The purity and recovery yield of CD4 and CD8 T cells in a positive selection is based on number conservation and elution. The elution efficiency process may be enhanced by optimizing the temperature, incubation time and free biotin buffer concentration.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

1. A method for manufacturing particles, comprising:
preparing a lipid compound;
combining a perfluorocarbon with the liquid compound; and
agitating the combination.

2. The method of claim 1, further comprising agitating the combination by one or more of centrifugation, sonication, homogenization or mechanical agitation.

3. The method of claim 1, further comprising agitating the combination to achieve a predetermined particle size distribution.

4. The method of claim 3, wherein the particle size distribution is in a range of from about 400 nm to about 300 microns.

5. The method of claim 1, further comprising combining different lipids in a sequence based on a characteristic of each lipid to prepare the lipid compound.

6. The method of claim 5, further comprising:
preparing a solution with a lipid solvent;
heating the solution; and
adding the different lipids to the solution in order of solubility.

7. The method of claim 1, wherein the lipid compound comprises one or more of DPPA, DPPC, DSPC, PEG40 Stearate, DSPE-mPEG(2000), DSPE-PEG(2000)-Biotin, DSPE-PEG-5000-Biotin, DSPE-PEG(2000)-Desthiobiotin, PBS buffer, glycerol, propyleneglycol, or DSPE-PEG(2000)-Maleimide.

8. The method of claim 1, wherein the perfluorocarbon is one or more of perfluoropentane, perfluorohexane, perfluorooctane, perfluorooctyl bromide, perfluorodichlorooctane, or perfluorodecalin.

9. The method of claim 1, further comprising functionalizing the particle with a linker.

10. The method of claim 9, wherein the linker is reversible.

11. The method of claim 9, wherein the linker comprises one or more of Avidin, Neutravidin, Streptavidin, Captavidin, biotin, desthiobiotin, an antibody, an aptamer or an oligomer.

12. The method of claim 1, further comprising applying a stabilizer or surfactant to the particles.

13. A particle manufactured according to any of claim 1.

14. A particle for use in cell selection, comprising:
a perfluorocarbon core; and
a lipid shell that covers at least a portion of the core;
wherein the lipid shell is functionalized with a linker.

15. The particle of claim 14, further comprising a plurality of the particles, wherein the particle size distribution of the particles is in a range of from about 400 nm to about 300 microns.

16. The particle of claim 14, wherein the lipid shell further comprises a combination of different lipids.

17. The particle of claim 16, wherein the lipid shell comprises one or more of DPPA, DPPC, DSPC, PEG40 Stearate, DSPE-mPEG(2000), DSPE-PEG(2000)-Biotin, DSPE-PEG-5000-Biotin, DSPE-PEG(2000)-Desthiobiotin, PBS buffer, glycerol, propyleneglycol, or DSPE-PEG(2000)-Maleimide.

18. The particle of claim 14, wherein the perfluorocarbon core comprises one or more of perfluoropentane, perfluorohexane, perfluorooctane, perfluorooctyl bromide, perfluorodichlorooctane, or perfluorodecalin.

19. The particle of claim 14, wherein the linker comprises one or more of Avidin, Neutravidin, Streptavidin, Captavidin, biotin, desthiobiotin, an antibody, an aptamer or an oligomer.

20. The particle of claim 14, further comprising a stabilizer or surfactant.

21. A method for separating target particles from a fluid, comprising:
receiving functionalized particles of claim 14 in the fluid in a chamber;
receiving target particles in the chamber;
permitting the target particles to bind with the functionalized particles;
applying an acoustic wave to the chamber to influence the functionalized particles to be collected or blocked by the acoustic wave.
